(19) United States
(12) Patent Application Publication
(10) Pub. No.: US 2025/0177205 A1
(43) Pub. Date: Jun. 5, 2025

(54) THIN-FILM OCULAR IMPLANT HAVING A CONTROL STRUCTURE FOR REGULATING AQUEOUS FLOW

(71) Applicant: Avisi Technologies, Inc., Redwood City, CA (US)

(72) Inventor: Jeffrey Lorne Emery, Emerald Hills, CA (US)

(21) Appl. No.: 18/970,063

(22) Filed: Dec. 5, 2024

Related U.S. Application Data
(60) Provisional application No. 63/606,472, filed on Dec. 5, 2023.

(51) Int. Cl. A61F 9/007

(52) U.S. Cl. CPC A61F 9/00781

(57) ABSTRACT

Disclosed herein is a device, system and method configured to safely regulate aqueous outflow in a patient's eye while dynamically maintaining a normal range of intraocular pressure. An example thin-film implant comprises a plurality of topographical features implemented on first and second surfaces of the implant. The topographical features include a network of interconnected channels configured to direct aqueous humor in a patient's eye to flow through and across the surfaces of the thin-film implant for lowering intraocular pressure. Multiple control structures are implemented on one or more selected locations of the first and second surfaces to regulate the aqueous humor in the patient's eye.

### CROSS REFERENCE TO RELATED APPLICATION

The present application claims the benefit of U.S. Provisional Patent Application Ser. No. 63/606,472 filed Dec. 5, 2023, the entire contents of each of which are incorporated by reference herein.

### BACKGROUND

Glaucoma is a group of eye conditions that damage the optic nerve. It is a leading cause of irreversible blindness for people over the age of 60 and affects more than 65 million people globally with projections to increase to 111.8 million by 2040. The most common form of glaucoma is primary open-angle glaucoma, in which aqueous humor cannot drain properly through the blocked trabecular meshwork. As the aqueous humor accumulates in the eye, it increases intraocular pressure (IOP) and damages the optic nerve, leading to slow, asymptomatic vision loss. Importantly, IOP remains the only modifiable risk factor for glaucoma.

Permanent glaucoma drainage devices have been developed to target different severities of disease: tube shunts mainly treat refractory or severe glaucoma, while minimally invasive glaucoma surgeries, angle-based procedures that do not result in subconjunctival filtration, are approved for mild-to-moderate glaucoma. Tube shunts are generally more effective over a 5-year period, and are increasingly used as a first-line filtration surgery, compared to the gold-standard incisional filtration surgery, trabeculectomy; however, tube shunts have a thick profile, remain invasive, and often reserved as a last line of defense. Meanwhile, subconjunctival microstents (e.g., Xen gel stent, Preserflo) have poor drainage-sustaining capability when compared to tube shunts, often requiring revision procedures like needling. Furthermore, both tube shunts and subconjunctival microstents rely on a single-lumen tube to drain aqueous humor to lower IOP. Single-lumen tubes are prone to occlusion with scar tissue and erosion through healthy tissue, which may necessitate revision surgery.

Accordingly, there is a need to provide a device, system and method configured to safely regulate aqueous outflow while dynamically maintaining a normal range of IOP.

### SUMMARY

Among other features, the present disclosure relates to a thin-film implant that lowers intraocular pressure. The thin-film implant comprises a first surface opposite a second surface; a plurality of topographical features on each of the first and second surfaces, wherein the plurality of topographical features include a plurality of interconnected channels configured to direct aqueous humor in a patient's eye to flow through and across the first and second surfaces of the thin-film implant for lowering intraocular pressure in the patient's eye; and a plurality of control structures implemented on one or more selected locations of the first and second surfaces to regulate the aqueous humor in the patient's eye.

In one embodiment, the plurality of control structures may include a plurality of gates for opening and closing flows of the aqueous humor in the patient's eye. Each gate may be configured to open or close in a number of degrees to control the flows of the aqueous humor, thereby reducing the intraocular pressure to a target range, wherein the number of degrees correspond to detected flow rate of the aqueous humor in the patient's eye.

In certain implementations, the plurality of control structures may be implemented across the first and second surfaces of the thin-film implant. In alternate embodiments, the control structures may be positioned near a distal end of an extension portion of the thin-film implant to form a gated entry for the aqueous humor in the patient's eye. In yet another embodiment, the plurality of control structures may be positioned in a region where an extension portion of the thin-film implant connects with a main body of the thin-film implant.

In one aspect, the plurality of control structures may be configured to open in response to detecting an elevated intraocular pressure in the patient's eye, and deflect toward closure in response to detecting a decreasing intraocular pressure in the patient's eye.

Further, each of the plurality of control structures may be configured to have a size and a position relative to each of the plurality of interconnected channels to achieve different pressure gradients across various portions of the thin-film implant. For example, a ratio between the plurality of control structures and the plurality of interconnected channels may be 1:1.

According to an embodiment, at least a portion of the control structures may include covers configured to prevent aqueous leaking or tissue ingrowth.

According to another embodiment, at least a portion of the control structures may include hinge portions, wherein each hinge portion associated with each control structure is configured to open or close in a number of degrees to control flows of the aqueous humor.

In addition, at least one of the plurality of gates has a vertical orientation or a horizontal orientation.

According to additional embodiments, at least one of the plurality of gates may have a thickened wall implemented on a selected side surface of a hexagonal microstructure of the thin-film implant. The thickened wall may be configured to form a live hinge to tune a dynamic resistance of the flows of the aqueous humor in the patient's eye.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTIONS OF THE DRAWINGS

The present disclosure will become more fully understood from the detailed description and the accompanying drawings, wherein:

FIG. 1 is a perspective view of a device according to one embodiment.

FIG. 2 is a close-up view of the device according to section A identified in FIG. 1.

FIG. 3 is a cross-sectional view of the device shown along line III-III in FIG. 2.

FIG. 4 is a perspective view of a device according to another embodiment.

FIG. 5A is a portion of a cross-sectional view of section A of the device shown in FIG. 4 according to one embodiment.

FIG. 5B is a portion of a cross-sectional view of section A of the device shown in FIG. 4 according to one embodiment.

FIG. 5C is a portion of a cross-sectional view of section A of the device shown in FIG. 4 according to one embodiment.

FIG. 6 is a diagram of a control structure implemented on a thin-film implant for regulating aqueous humor in a patient's eye, according to an example embodiment of the present disclosure.

FIG. 7 is a side view of the control structure of FIG. 6, according to an example embodiment of the present disclosure.

FIG. 8 is a diagram of a plurality of control structures implemented on selected portions of a thin-film implant, according to an example embodiment of the present disclosure.

FIGS. 9A and 9B illustrate that a plurality of control structures are implemented at two examples locations of a thin-film implant, according to an example embodiment of the present disclosure.

FIG. 10 is a diagram of a side view of a closure or openness of a control structure having varying degrees, according to an example embodiment of the present disclosure.

FIG. 11 is a diagram of multiple control structures implemented on a thin-film implant at different locations, according to an example embodiment of the present disclosure.

FIGS. 12A and 12B illustrate a cover for a recess implemented a control structure, according to an example embodiment of the present disclosure.

FIG. 13 is a side view of a control structure having a hinge portion, according to an example embodiment of the present disclosure.

FIGS. 14A and 14B illustrate a vertical orientation and a horizontal orientation of a control structure, respectively, according to an example embodiment of the present disclosure.

FIG. 15 is a side view of the horizontal orientation of the control structure of FIG. 14B, according to an example embodiment of the present disclosure.

FIG. 16 illustrates a control structure having thickened walls for gate construction, according to an example embodiment of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

It should be appreciated that the device, system and method of the present disclosure may be utilized in any one or more medical or surgical procedures that involve fragile thin-film like implant such as, for example cardiac surgery, anastomosis procedures, non-surgical procedures, endoscopic procedures, non-invasive procedures, invasive procedures, port-access procedures, fluoroscopic procedures, beating heart surgery, vascular surgery, neurosurgery, electrophysiology procedures, diagnostic and therapeutic procedures, ablation procedures, ablation of arrhythmias, endovascular procedures, treatment of one or more organs and/or vessels, cardiograms, pharmacological therapies, drug delivery procedures, delivery of biological agents, gene therapies, cellular therapies, cancer therapies, radiation therapies, genetic, cellular, tissue and/or organ manipulation or transplantation procedures, coronary angioplasty procedures, placement or delivery of coated or uncoated stents, placement of cardiac reinforcement devices, placement of cardiac assistance devices, atherectomy procedures, atherosclerotic plaque manipulation and/or removal procedures, emergency procedures, cosmetic procedures, reconstructive surgical procedures, biopsy procedures, autopsy procedures, surgical training procedures, birthing procedures, congenital repair procedures, and medical procedures that require manipulation and delivery of one or more fragile thin-film like implant into a surgical site.

In one embodiment, as will be described fully below, the present disclosure relates to a thin-film based ocular implant such as for treatment of glaucoma. A glaucoma drainage implant is a small device (i.e., a thin-film device) placed in an eye of a patient to treat glaucoma. Most glaucoma patients have abnormally high IOP due to the patient's inability to drain excessive aqueous humor from the anterior chamber of the eye through the trabecular meshwork. If not reduced with adequate treatment, high IOP will continuously damage the optic nerve as the disease progresses, leading to loss of vision or even total blindness. During a glaucoma implant surgery, a tiny drainage hole may be made in the sclera of the patient's eye (the white part of the eye). This opening allows fluid to drain out of the eye under the delicate membrane covering the eyeball known as the conjunctiva. Locally applied medications or injections may be used to keep the hole open and a thin-film glaucoma drainage device is positioned on the outside of the eye under the conjunctiva to drain excessive fluid out of the eye and into a place where the capillaries and lymphatic system of the patient reabsorb it back into the body, thereby lowering the intraocular pressure.

To reduce scaring and post-operative patient discomfort, it is desired to make as small as incision of the conjunctiva as possible, ideally less than 3 millimeters (“mm”). As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the structure be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Unless otherwise specified, all percentages and amounts expressed herein and elsewhere in the specification should be understood to refer to percentages by weight. The amounts given are based on the weight of the material. According to the present application, the term “about” means+/−5% of the reference value. According to the present application, the term “substantially free” means less than about 0.1 wt. % based on the total of the referenced value.

A “subject” herein may be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

### Treatment Device Embodiment

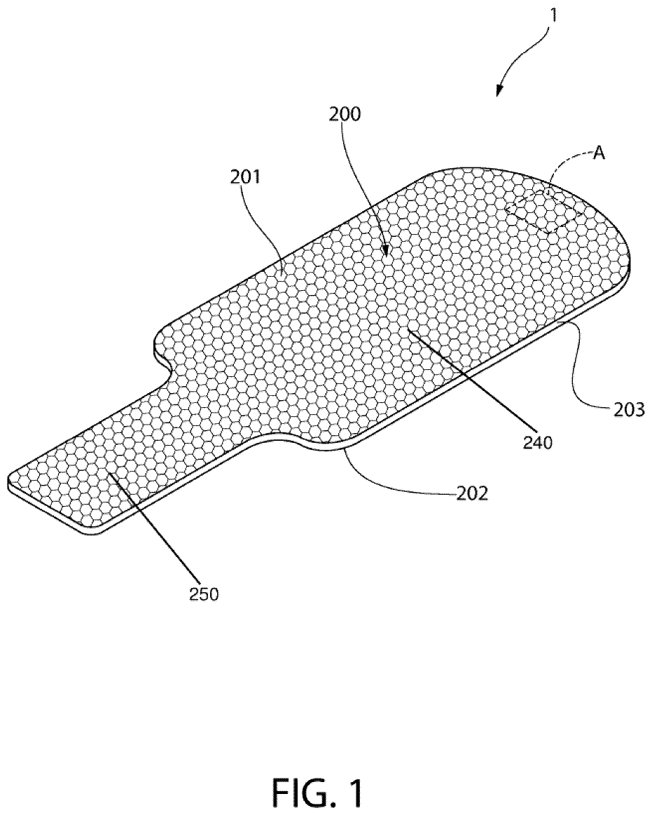

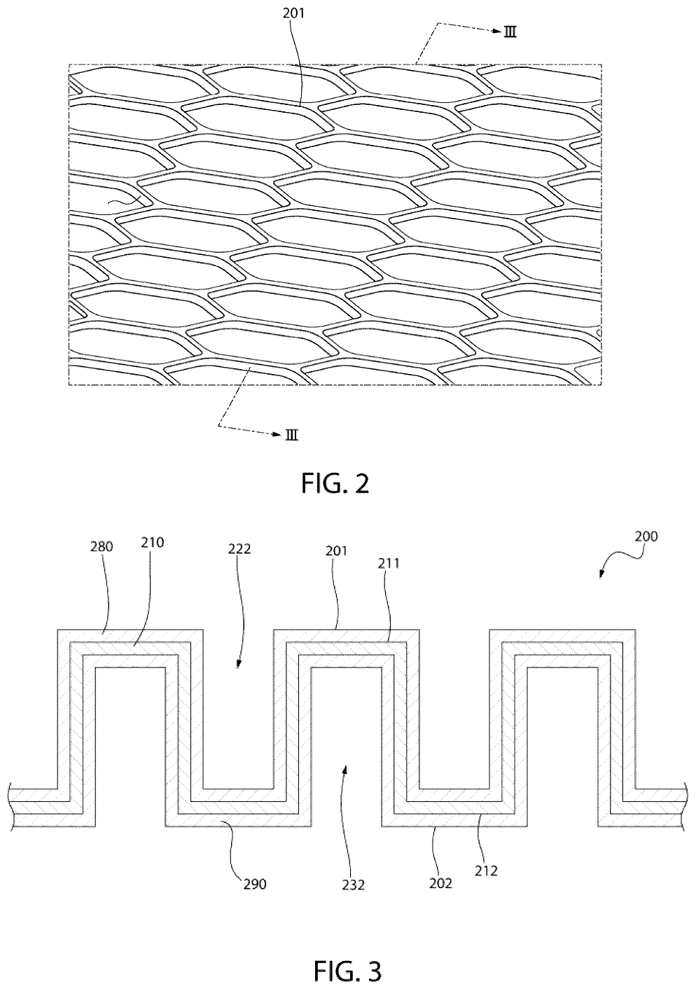

Referring to FIGS. 1-3, a treatment device 1 includes a plate structure 200, or simply plate, having a first major exposed surface 201 opposite a second major exposed surface 202 as well as side surface 203 extending there-between. The plate structure 200 can comprise an extension portion 250 and a main body portion 240.

The plate structure 200 can be formed of any material with appropriate characteristics for implantation and treatment. In some embodiments, the plate structure 200 can be formed of a metal, polymer, ceramic (e.g., aluminum oxide), other composite material, or a combination thereof. Metals can include, but are not limited to aluminum, titanium, zinc, platinum, tantalum, copper, nickel, rhodium, gold, silver, palladium, chromium, iron, indium, ruthenium, osmium, tin, iridium, or combinations, and alloys thereof. In some embodiments, alloys can include steel and nickel titanium such as Nitinol.

Polymers or polymer materials used to form plate structure 200 can include any of the polymers described herein.

Composites such as silicon composites can also be used. In one embodiment, a composite can include silicon nitride (Si₃N₄). The silicon nitride can have any known crystalline structure such as, but not limited to, trigonal α-Si₃N₄, hexagonal β-Si₃N₄, or cubic γ-Si₃N₄.

The plate structure 200, or plate, can have a thickness ranging from about 1 nm to about 1,000 nm, from about 1 nm to about 500 nm, from about 1 nm to about 400 nm, from about 100 nm to about 1,000 nm, from about 200 nm to about 1,000 nm, from about 300 nm to about 1,000 nm, from about 400 nm to about 1,000 nm, from about 1 nm to about 900 nm, from about 1 nm to about 800 nm, from about 1 nm to about 700 nm, from about 1 nm to about 600 nm, from about 300 nm to about 500 nm, from about 300 nm to about 600 nm, from about 400 nm to about 600 nm, from about 200 nm to about 600 nm, from about 200 nm to about 500 nm, or from about 50 nm to about 800 nm.

The plate structure 200 may comprise a multi-directional plate 210 comprising a first major surface 211 opposite a second major surface 212. The multi-directional plate 210 may form a plurality of topographical features (for example, a repeating honeycomb pattern) on each of the first major surface 211 and the second major surface 212. Each of the first and second topographies may independently comprise a plurality of channels 232 and/or a plurality of open-cells 222.

The plurality of channels 232 may be interconnected and can form a network of channels. The channels may be open or closed, allowing fluid to readily enter each channel of plurality of channels 232 and flow through it. The network may comprise intersecting channels in any suitable configuration to best help promote the flow of fluid across the plate structure 200 via the plurality of channels 232. In one embodiment, the channels 232 may be configured to form hexagonal patterns. Once treatment device 1, illustrated in FIG. 1, is implanted, fluid (e.g., aqueous humor) may be driven by a pressure gradient to flow through the channels and across the surface of plate structure 200.

In some embodiments, the channels 232 can include a ribbing pattern. The ribbing pattern and/or the geometry of the channels in the plate can be varied based on different severities of disease (e.g., mild, moderate, or severe glaucoma). In one embodiment, larger or smaller channels can be used to decrease intraocular pressure by different amounts. Changing intraocular pressure by a lower amount can decrease risk of hypotony (a condition that can exist if intraocular pressure is reduced too much) and increase efficacy at lowering pressure to a target level. In some embodiments, a device as described herein with smaller channels can decrease flow and decrease risk of hypotony. Likewise, larger channels can increase flow and allow the device to reduce intraocular pressure to a lower level.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210.

The first coating 280 may have a thickness ranging from about 0.1 μm to about 10 μm or about 0.1 μm to about 2 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

The plate structure 200 may further comprise a second coating 290 applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The second coating 290 may have a thickness ranging from about 0.1 μm to about 10 μm or about 0.1 μm to about 1 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

In some embodiments, the plate structure 200 may comprise only the first coating 280—i.e., no second coating. In other embodiments, the plate structure 200 may comprise only the second coating 290—i.e., no first coating. In other embodiments, the plate structure 200 may comprise the first coating 280 and the second coating 290, whereby the first and second coatings overlap to fully encapsulate the multi-directional plate 210. In such embodiments, the side surface 203 of the plate structure 200 may comprise at least one of the first coating 280 and the second coating 290.

In some embodiments, the first and second coating, and any edge coating, can be thicker than the plate itself. In some embodiments, the coating thickness can be one, two or three orders of magnitude thicker than the plate structure. However, in other embodiments, the plate can be thicker than each coating or the additive thickness of the two coatings.

Coatings described herein can be applied by any suitable deposition method, such as but not limited to, physical vapor deposition, chemical vapor deposition, atomic layer deposition, spray coating, spin coating, self-assembly, dip coating, or brushing.

The first coating 280 may be applied to the first major surface 211 by any suitable deposition method. In a non-limiting example, the first coating 280 may be applied to the first major surface 211 by chemical vapor deposition, physical vapor deposition, or plasma-enhanced chemical vapor deposition. In another non-limiting example, the first coating 280 may be applied to the first major surface 211 by atomic layer deposition. In another non-limiting example, the first coating 280 may be applied to the first major surface 211 by spray coating. In another non-limiting example, the first coating 280 may be applied to the first major surface 211 by dip coating. In another non-limiting example, the first coating 280 may be applied to the first major surface 211 by brushing.

The second coating 290 may be applied to the second major surface 212 by any suitable deposition method. In a non-limiting example, the second coating 290 may be applied to the second major surface 212 by chemical vapor deposition, physical vapor deposition, or plasma-enhanced chemical vapor deposition. In another non-limiting example, the second coating 290 may be applied to the second major surface 212 by atomic layer deposition. In another non-limiting example, the second coating 290 may be applied to the second major surface 212 by spray coating. In another non-limiting example, the second coating 290 may be applied to the second major surface 212 by dip coating. In another non-limiting example, the second coating 290 may be applied to the second major surface 212 by brushing.

The first coating 280 may be the same as the second coating 290. The first coating 280 and the second coating 290 may be different. The first coating 280 may be hydrophilic. The first coating 280 may be hydrophobic. The first coating 280 may be lipophilic. The first coating 280 may be lipophobic. The second coating 290 may be hydrophilic. The second coating 290 may be hydrophobic. The second coating 290 may be lipophilic. The second coating 290 may be lipophobic. Each of the first and second coatings 280, 290 may independently be continuous. Each of the first and second coatings 280, 290 may independently be discontinuous. In some embodiments, the first and second coatings 280, 290 may both be hydrophobic. In some embodiments, the first and second coatings 280, 290 may both be hydrophilic. In some embodiments, the first and second coatings 280, 290 may both be lipophilic or lipophobic.

The first coating 280 may be organic. The first coating 280 may be inorganic. The second coating 290 may be organic. The second coating 290 may be inorganic.

In some embodiments, the first coating 280 is hydrophilic and the second coating 290 is hydrophobic. In some embodiments, the first coating 280 is hydrophilic and the second coating 290 is hydrophilic. Having at least one of the first and/or second coating 280, 290 be hydrophobic may help prevent the treatment device 1 from inadvertently sticking to tissue during implantation.

In some embodiments, a purpose of a first and/or second coating is to increase the toughness of the device. Also, a first and/or second coating can increase biocompatibility of the device and/or decrease scarring by decreasing tissue and/or fibroblast adhesion. In some embodiments, the coatings described herein are hydrophobic and decrease tissue adhesion. In some embodiments, tissue adhesion can be reduced by greater than about 10%, greater than about 60%, greater than about 70%, greater than about 80%, greater than about 90%, greater than about 95%, greater than about 96%, greater than about 97%, greater than about 98%, or greater than about 99% when compared to an uncoated plate.

In a non-limiting embodiment, the first and/or second coating may comprise a polymer, such as a parylene polymer (poly(para-xylylene)) or a derivative thereof. In other embodiments, the first and/or second coating can include aluminum oxide, a biocompatible film, a porous coating, or a lubricious coating. In one embodiment, the parylene polymer is a chlorine modified poly(para-xylylene), or a fluorine modified poly(para-xylylene). In one embodiment, the parylene polymer can be parylene C, parylene D, parylene N, a derivative thereof or a combination thereof. In other embodiments, the first and/or second coating can include aluminum oxide.

In other embodiments, other polymer(s) can be used in addition to, in combination with, or instead of a parylene polymer and/or aluminum oxide. In some embodiments, other polymeric materials can include, but are not limited to rubber, synthetic rubber, silicone polymers, thermoplastics, thermosets, polyolefins, polyisobutylene, acrylic polymers, ethylene-co-vinylacetate, polybutylmethacrylate, vinyl halide polymers (for example, polyvinyl chloride), polyvinyl ethers (for example, polyvinyl methyl ether), polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides (for example, Nylon 66 and polycaprolactam), alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene (for example, Teflon), poly(ether-ether-ketone), poly lactides such as PLA, PLGA, PLLA, derivatives thereof, or combinations thereof.

The resulting treatment device 1 may comprise the first plurality of channels 222 present on the first exposed major surface 201 of the plate structure 200, wherein the first plurality of channels 222 are hydrophilic due to the presence of the first coating 280. The resulting treatment device 1 may comprise the second plurality of channels 232 present on the second exposed major surface 202 of the plate structure 200, wherein the second plurality of channels 232 are hydrophilic due to the presence of the second coating 290. As discussed, the hydrophilic channels may promote fluid flow through the channels after the treatment device 1 has been implanted into a subject's eye.

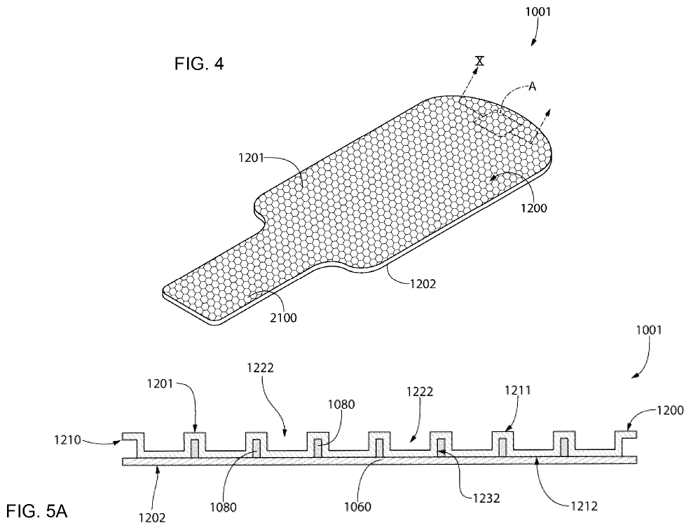

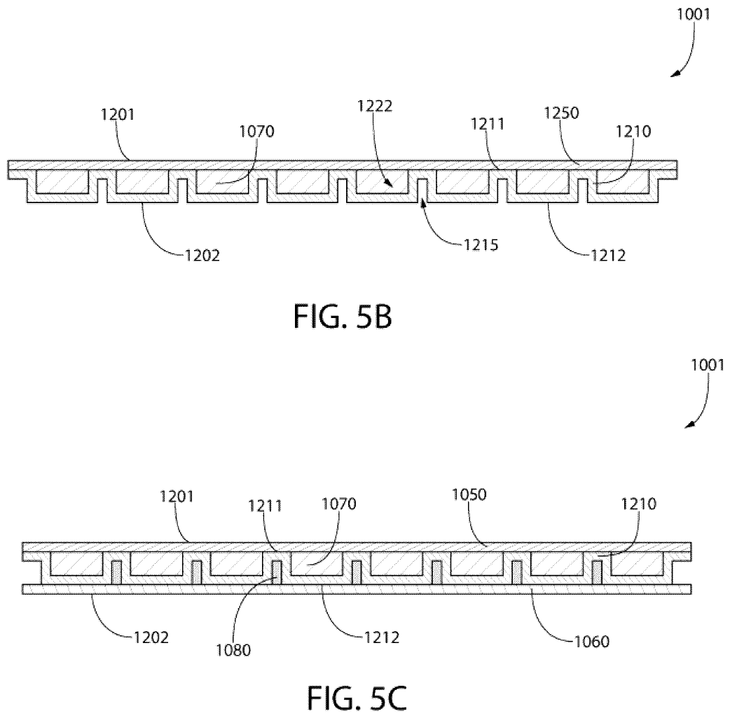

Referring to FIGS. 4, 5A, 5B, and 5C, generally, a treatment device 1001 is illustrated in accordance with another embodiment. The treatment device 1001 is similar to the treatment device 1 except as described herein below. The description of the treatment device 1 above generally applies to the treatment device 1001 described below except with regard to the differences specifically noted below. A similar numbering scheme will be used for the treatment device 1001 as with the treatment device 1 except that a “1000” series of numbering will be used.

The treatment device 1001 comprises a plate structure 1200 having a first exposed major surface 1201 that is opposite a second exposed major surface 1202. The plate structure 1200 may comprise a multi-directional plate 1210 comprising a first major surface 1211 opposite a second major surface 1212. The multi-directional plate 1210 may form a plurality of topographical features (for example, a repeating honeycomb pattern) on each of the first major surface 1211 and the second major surface 1212. Each of the first and second topographies may independently comprise a plurality of channels 1232 and/or a plurality of open-cells 1222.

Referring now to FIG. 5B, the plate structure 1200 may comprise a first drug-treatment delivery component 1070 present in the open voids created by the first topography formed by the first exposed surface 1211 of the multi-directional plate 1210. Specifically, the first delivery component 1070 may be present in the open voids created by the open-cells 1222 of first topography formed by the first major surface 1211 of the multi-directional plate 1210.

The first drug-treatment delivery component 1070 may comprise one or more active agents such as, but not limited to therapeutic and/or pharmacological components. The first drug-treatment delivery component 1070 may occupy some, all, or substantially all of the free volume present in the open-cells 1222 formed by the first topography.

In other embodiments, active agents can include any compound or drug having a therapeutic effect in a subject. Non limiting active agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, steroids, proteasome inhibitors, antibiotics, anti-inflammatories, anti-sense nucleotides, transforming nucleic acids, messenger ribonucleic acids, IOP lowering drugs, prostaglandins, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, cells, stem cells, liposomes, anti-metabolites such as mitomycin-C, combinations thereof, prodrugs thereof, pharmaceutical salts thereof, derivatives thereof, and the like.

The treatment device 1001 may further comprise a first coating 1050 applied to a first major surface 1211 of the multi-directional plate 1210. The first coating 1050 may cover both a first major surface 1211 of the multi-directional plate 1210 as well as a first drug-treatment delivery component 1070 that is present in the open-cells 1222 formed into the first major surface 1211 of the multi-directional plate 1210. The first coating 1050 may be in the form of a continuous film. The first coating 1050 may be flat. In other embodiments, the first coating 1050 may be conformal to the underlying pattern formed by the multi-directional plate 1210 and the first delivery component 1070.

Referring now to FIG. 5A, the plate structure 1200 may comprise a second drug-treatment delivery component 1080 present in the open voids created by the second topography formed by the second exposed surface 1212 of the multi-directional plate 1210. Specifically, the second delivery component 1080 may be present in the open voids created by the open-channels 1232 of the second topography formed by the second major surface 1212 of the multi-directional plate 1210. The second delivery component 1080 may be the same or different from the first delivery component 1070.

The second drug-treatment delivery component 1080 may comprise one or more therapeutic and/or pharmacological components—including but not limited to anti-inflammatory agents, steroids, antibiotics, analgesics. The second delivery component 1080 may occupy some, all, or substantially all of the free volume present in the channels 1232 formed by the first topography.

The treatment device 1001 may further comprise a second coating 1060 applied to a second major surface 1212 of the multi-directional plate 1210. The second coating 1060 may cover both the second major surface 1212 of the multi-directional plate 1210 as well as the second delivery component 1080 that is present in the open-channels 1232 formed into the second major surface 1212 of the multi-directional plate 1210. The second coating 1060 may be in the form of a continuous film. The second coating 1060 may be flat. In other embodiments, the second coating 1060 may be conformal to the underlying pattern formed by the multi-directional plate 1210 and the second delivery component 1080.

The second coating 1060 may be the same or different than the first coating 1050. For each of the first and the second coatings 1050, 1060, the resulting film may be formed from a slow-release material that dissolves slowly after exposure to aqueous humor or other biological fluids, thereby releasing the first delivery component 1070 from the channels 1232 of the treatment device 1001 after it has been implanted into a subject.

Referring now to FIG. 5C, in other embodiments, the treatment device 1001 may comprise both the first and the second drug-treatment delivery components 1070, 1080, as well as the first and the second coatings 1050, 1060 to encapsulate the first and second delivery components 1070, 1080.

In other embodiments, the plate structure 1200 may comprise at least one of the first coating 1050 and/or the second coating 1060 without the presence of the first and/or second delivery components 1070, 1080. In such embodiments, the first coating 1050 and/or the second coating 1060 may form a film that covers the open cells 1222 and/or the open channels 1232 created by the multi-directional plate.

The presence of the films resulting from the first and/or the second coating 1050, 1060 may enhance the overall strength of the resulting treatment device. Specifically, layered structure(s) of the films formed by the first and second coatings 1050, 1060, which are bonded to the first and second major surfaces 1211, 1212 of the multi-directional plate 1210, provide added mechanical integrity to the resulting treatment device.

Beyond achieving the baseline flexibility to conform to curvature of the eye, the addition of the first and/or second coatings 1050, 1060 may provide a mechanism that allows the overall treatment device to match the elastic modulus of surrounding tissues (e.g., conjunctival and scleral tissues) to maximize biocompatibility or biointegration. Findings in brain implant research confirm that the flexibility of implants in soft tissue improves compliance of the implant with microscale movements of surrounding tissue and reduces tissue displacement and trauma as well as facilitates implantation of the treatment device.

### Control Structure For Regulating Aqueous Flow

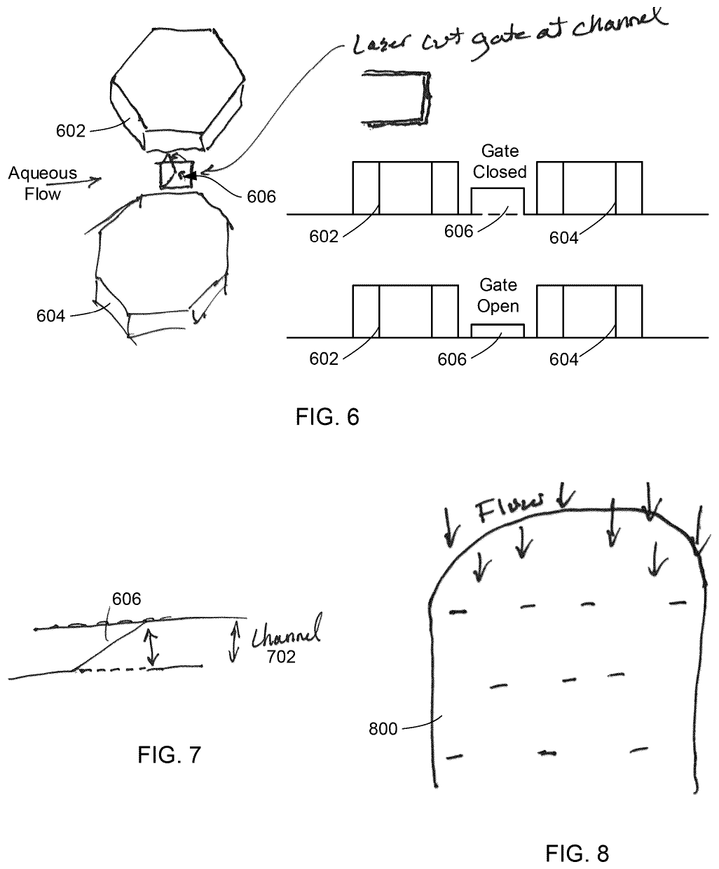

In accordance with aspects of the present disclosure, a control structure (e.g., a laser cut gate) may be implemented at selected locations of a plate structure described above with respect to FIGS. 1-4, 5A, 5B, and 5C to regulate aqueous humor in a patient's eye. As shown in FIGS. 6 and 7, a laser cut gate 606 may be positioned at, e.g., a channel 702 formed between two adjacent hexagonal microstructures 602, 604 of a plate structure. As will be described fully below, the gate 606 may be configured to open or close in various degrees to control the aqueous flow through the channel 702.

In one aspect, the locations of the control structures may be determined and arranged on a plate structure in a way to optimize the resistance to the flow aqueous humor from a patient's eye, thereby reducing the IOP to a target range. For example, there may be one control structure implemented within each channel formed between adjacent hexagonal microstructures, as shown in FIG. 6.

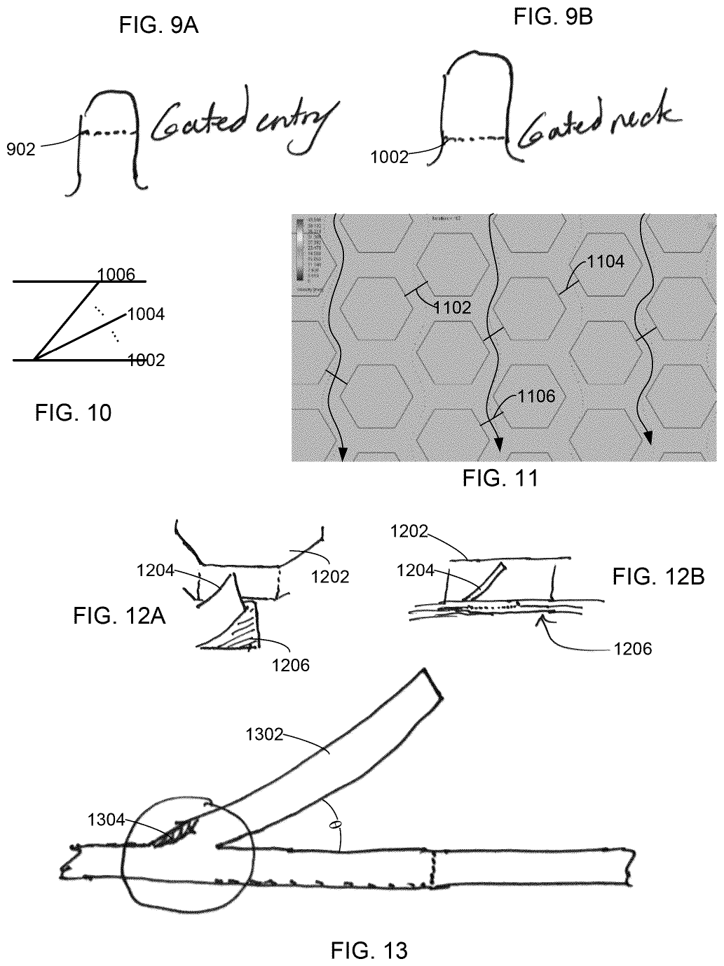

For another example, a plurality of control structures may be implemented across the entire extension portion (e.g., similar to the extension portion 250 depicted in FIG. 1) of a plate structure 800, as shown in FIG. 8. According to one embodiment, as shown in FIG. 9A, a plurality of control structures 902 may be positioned near a distal end of the extension portion of a plate structure to form a gated entry for aqueous flow in a patient's eye. According to another embodiment, as shown in FIG. 9B, a plurality of control structures 1002 may be positioned in a region where the extension portion connects with the main body (e.g., similar to the main body portion 240 depicted in FIG. 1) of a plate structure. It may be optimal to regulate the flow of aqueous at the entry of the thin-film shunt, before the flow develops significant velocity and is at a higher pressure, or further downstream, when the flow as developed velocity within the channels and is at a lower pressure.

In certain aspects, the control structure of the present disclosure may function as a passive tuning gate with its angulation tuned to regulate dynamically changing IOP in the anterior chamber. For example, in response to detecting an elevated IOP or flow in a patient's eye, the gate may be configured to open. On the other hand, in response to detecting a decreasing IOP, the gate may be configured to deflect toward closure.

The control structure may be configured to generate resistance to flow via several design options. For example, the closure or openness of the gate may be configured to have varying degrees. As shown in FIG. 10, the gate may have a fully open state 1002 that may be approximately proportional to a high rate of aqueous humor outflow from the anterior chamber of the eye. The gate may gradually move to an intermediate state 1004 while upstream velocity of the aqueous flow is decreasing. Once the IOP has been determined to reach a selected threshold value, the gate may be fully closed (fully close state 1006 depicted in FIG. 10). It should be appreciated that the gate may have multiple intermediate positions between the illustrated states 1002, 1004, and 1006 of FIG. 10 for regulating the aqueous flow purposes.

According to another example, the size (height and width) and position of each gate may be determined and implemented relative to each channel to achieve different pressure gradients across various portions of the plate structure. As shown in FIG. 11, multiple gates 1102, 1104, 1106 may be positioned in selected channels to generate different pathways and resistance to aqueous outflows. The number of gates may be determined based upon the number of channels. Generally, it may be preferred to have a 1:1 ratio of gates per channel, but a higher or lower ratio may be implemented. This could depend upon a variety of factors, such as the individual resistance each gate provides, the geometry of both the channels and gates, and the relative resistance impact provided by each gate, and if significant mixing of flow occurs along the shunt between the array of channels.

According to an alternate embodiment, the manufacturing consequences of using cutting techniques, such as laser, may result in holes from which the gate is created. If holes are undesired, such as causing a leak pathway for aqueous, then a cover element may be integrated into the device manufacturing. Referring to FIGS. 12A and 12B, a gate recess cover 1206 may be implemented for a gate 1204 adjacent a hexagonal microstructure 1202. In some embodiments, if the construction method of the gate(s) results in a hole within the recess of the gate, this hole may become a leak point for aqueous humor and/or a path in which tissue may grow and obstruct the deflection or closure of the gate. A recess cover may be integrated as a separate member such as a coating on the bottom of the device, such that the cover may prevent aqueous leaking or tissue ingrowth.

FIG. 13 illustrates a tuning gate 1302 with a hinge portion 1304. The torsional stiffness of the gate 1302 may be determined by a live hinge geometry and material. Further, the initial configuration of the gate (e.g., an initial angle θ between the gate 1302 and the horizontal surface of the plate structure) may be determined and formed via plastic deformation applied in manufacturing. The stiffness of the hinge portion 1304 may be adjusted or tuned in manufacturing by selective ablation or material removal at base, such as with laser and/or etching.

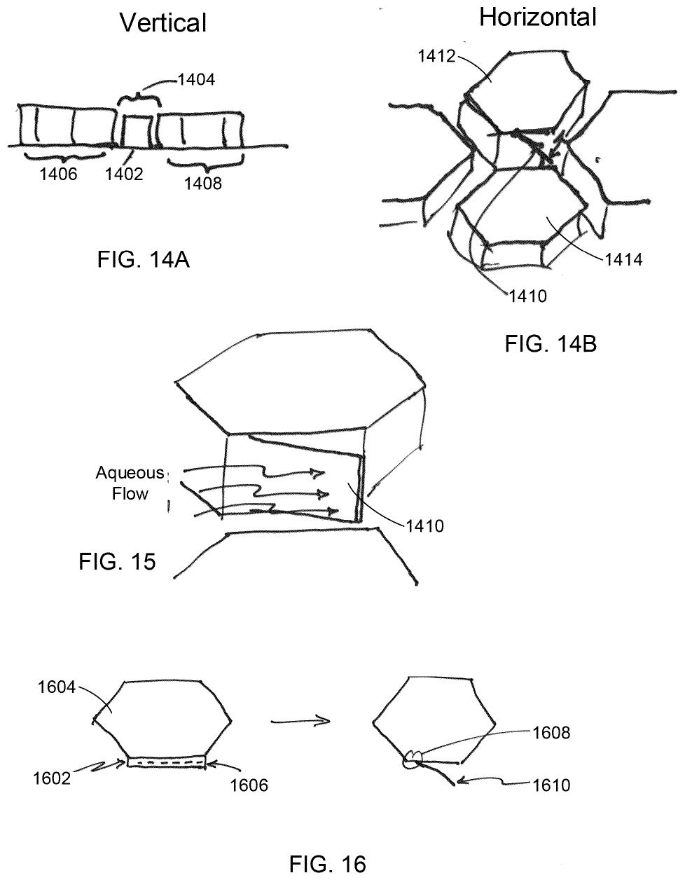

In yet another aspect, the control structure of the present disclosure may have different gate orientations. Referring to FIG. 14A, a vertical gate 1402 may be implemented in the channel 1404 between two adjacent hexagonal microstructures 1406, 1408. FIG. 14B illustrates an alternate embodiment where a horizontal gate 1410 is implemented in a channel formed between two adjacent hexagonal microstructures 1412, 1414. Such a horizontal gate 1410 may be configured to be partially or completely obstruct aqueous flow, as shown in FIG. 15.

According to further embodiments, the control structure of the present disclosure may have thickened walls for gate construction. As shown in FIG. 16, a thickened wall 1602 may be implemented on a selected side surface of a hexagonal microstructure 1604. Next, laser cutting and any other suitable cutting techniques along dotted line 1606 may be carried out on the thickened wall 1602 with some materials left in a region where a live hinge 1608 of a gate 1610 is created. As a result, the gate 1610 is created with partial wall thickness.

The following table shows various state conditions indicating how live hinge gates affect aqueous flow, where P represents pressure, Q refers to flow rate and R refers to flow resistance.

| P | Q | Gate Resistance | Downstream | Anterior Chamber Effect |
|---|---|---|---|---|
| Hi | Hi | Hi → Lo | Increasing Q | Decreasing P |
| Med | Med | Transition to medium | ~Q | Steady State |
| Lo | Lo | Transition to high | Decreasing Q | Increasing P |

As shown in the table, dynamic resistance of aqueous flow may be tuned by the control structure (e.g., live hinge gates) of the present disclosure to adapt to lower R when P is high and higher R when is low. The control structure may function as an effective shunt for high IOP and mitigate low IOP by decreasing aqueous outflow resistance automatically. As a result, the control structure may smooth out the profile of IOP to reduce risk of hypotony (excessively low IOP) while effectively shorting flow from anterior chamber to reduce high IOP.

According to an additional embodiment, the control structure of the present disclosure may be activated externally by a physician for example, either with laser or magnets to allow for more or less outflow based on treatment goals. Flow modulation via common ophthalmic lasers may be achieved by the incorporation of temperature-sensitive materials such as Nitinol in the gate structures, such that the gate structures may flex in response to the laser-generated heat and transition from one position to another to regulate flow to achieve the target IOP. Similarly, control structures incorporating paramagnetic materials may also be incorporated to facilitate post-operative flow adjustment by exposing the patient's eye to a magnetic field to transition the gates from one position to another. For both implementations, a ratchet and/or detent mechanism may be incorporated so that once the laser-induced heat or magnetic field is removed, the gate structures may retain the new configuration and the new flow resistance may be permanently altered.

In yet another embodiment, a sensor system (not shown) may be implemented to actively monitor and control flow of aqueous throughout the thin-film implant. Materials for the sensors may be biocompatible and corrosion-resistant (e.g., platinum, gold, graphene, or conductive polymers). Depending upon the parameter to monitor, a number of different sensors may be selected, such as micro-paddle wheels, hot-wire anemometers, or MEMS-based flow sensors, capacitive or piezoresistive pressure sensors, and ion-sensitive field-effect transistors (ISFETs) or optical sensors (pH/chemical sensors). Any suitable microfabrication techniques (e.g., photolithography, etching, or 3D printing) may be employed to make the sensor(s) compatible with the microstructure of the thin-film implant. In integrating the sensor system, direct deposition techniques such as sputtering, chemical vapor deposition (CVD), or inkjet printing may be used to deposit sensor materials directly onto the thin-film surface. In another embodiment, pre-fabricated micro-sensors may be attached to the thin-film implant using adhesive or thermal bonding while ensuring minimal impact on the microstructure or flow regulation properties. In an additional embodiment, one or more sensors may be embedded within or alongside the microstructures using soft lithography or laser ablation. Further, sensors may be placed at critical points where flow regulation is most sensitive or where feedback is needed while ensuring that the sensors do not obstruct or alter the designed flow dynamics, such as at the tip of implant where it communicates to the anterior chamber. Flexible and low-profile conductive traces (e.g., printed silver or gold lines) may be used to connect sensors to external circuits, according to certain implementations. Wireless power transfer (e.g., inductive coupling) or thin-film batteries may be utilized to minimize bulk. For encapsulation and protection purposes, in some embodiments, a thin, transparent, and waterproof coating (e.g., parylene, PDMS, or epoxy) may be applied to protect the sensors from water and mechanical damage. Such coating may not interfere with sensor functionality, especially for chemical or optical sensors. In further embodiments, wireless communication modules like Bluetooth, NFC, or LoRa may be incorporated into the sensor system for real-time monitoring without physical connections. Flexible antennas may be printed directly on the thin film if space is limited. One or more sensors may be additionally combined with control electronics, such as microcontrollers or custom ASICs, to process and act on sensor data in real-time. If active flow regulation may be needed, one or more actuators or valves may be integrated based on sensor feedback.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Unless otherwise indicated, all numbers expressing quantities and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A thin-film implant that lowers intraocular pressure, the thin-film implant comprising:
a first surface opposite a second surface;
a plurality of topographical features on each of the first and second surfaces, wherein the plurality of topographical features include a plurality of interconnected channels configured to direct aqueous humor in a patient's eye to flow through and across the first and second surfaces of the thin-film implant for lowering intraocular pressure in the patient's eye; and
a plurality of control structures implemented on one or more selected locations of the first and second surfaces to regulate the aqueous humor in the patient's eye.

2. The thin-film implant of claim 1, wherein the plurality of control structures include a plurality of gates for opening and closing flows in the plurality of interconnected channels of the aqueous humor in the patient's eye.

3. The thin-film implant of claim 2, wherein each of the plurality of gates is configured to open or close in a number of degrees to control the flows of the aqueous humor, thereby reducing the intraocular pressure to a target range, wherein the number of degrees correspond to detected flow rate of the aqueous humor in the patient's eye.

4. The thin-film implant of claim 1, wherein the plurality of control structures are implemented across the first and second surfaces.

5. The thin-film implant of claim 1, wherein the plurality of control structures are positioned near a distal end of an extension portion of the thin-film implant to form a gated entry for the aqueous humor in the patient's eye.

6. The thin-film implant of claim 1, wherein the plurality of control structures are positioned in a region where an extension portion of the thin-film implant connects with a main body of the thin-film implant.

7. The thin-film implant of claim 1, wherein the plurality of control structures are configured to open in response to detecting an elevated intraocular pressure in the patient's eye, and deflect toward closure in response to detecting a decreasing intraocular pressure in the patient's eye.

8. The thin-film implant of claim 1, wherein each of the plurality of control structures is configured to have a size and a position relative to each of the plurality of interconnected channels to achieve different pressure gradients across various portions of the thin-film implant.

9. The thin-film implant of claim 1, wherein a ratio between the plurality of control structures and the plurality of interconnected channels is 1:1.

10. The thin-film implant of claim 1, wherein at least a portion of the control structures include covers configured to prevent aqueous leaking or tissue ingrowth.

11. The thin-film implant of claim 1, wherein at least a portion of the control structures include hinge portions, wherein each hinge portion associated with each control structure is configured to open or close in a number of degrees to control flows of the aqueous humor.

12. The thin-film implant of claim 2, wherein at least one of the plurality of gates has a vertical orientation.

13. The thin-film implant of claim 2, wherein at least one of the plurality of gates has a horizontal orientation.

14. The thin-film implant of claim 2, wherein at least one of the plurality of gates has a thickened wall implemented on a selected side surface of a hexagonal microstructure of the thin-film implant.

15. The thin-film implant of claim 14, wherein the thickened wall is configured to form a live hinge to tune a dynamic resistance of the flows of the aqueous humor in the patient's eye.

16. The thin-film implant of claim 1, wherein at least a portion of the plurality of control structures are made of temperature-sensitive materials that are activatable by laser-induced heat to transition from one position to another to regulate the aqueous humor in the patient's eye.

17. The thin-film implant of claim 1, wherein at least a portion of the plurality of control structures are made of paramagnetic materials that are activatable by a magnetic field to transition from one position to another to regulate the aqueous humor in the patient's eye.

18. The thin-film implant of claim 1, further comprising a sensor system implemented on at least one of the first and second surfaces to monitor and control the aqueous humor in the patient's eye through the plurality of interconnected channels.

19. The thin-film implant of claim 18, wherein the sensor system includes at least one sensor selected from a flow sensor, a pressure sensor, or a pH/chemical sensor.

20. The thin-film implant of claim 19, wherein the at least one sensor is placed at a tip portion of the thin-film implant where it communicates to an anterior chamber of the patient's eye.
